(19) United States
(12) Patent Application Publication
(10) Pub. No.: US 2018/0311057 A1
(43) Pub. Date: Nov. 1, 2018

(54) INFLATABLE STENT

(71) Applicant: BOSTON SCIENTIFIC LIMITED, Hamilton (BM)

(72) Inventors: MEDHA TYAGI, Delhi (IN); JAYANT SITARAM KARVE, Bangalore (IN)

(73) Assignee: BOSTON SCIENTIFIC LIMITED, Hamilton (BM)

(21) Appl. No.: 15/962,726

(22) Filed: Apr. 25, 2018

Related U.S. Application Data
(60) Provisional application No. 62/490,538, filed on Apr. 26, 2017.

(51) Int. Cl. A61F 2/91; A61F 2/915; A61F 2/966

(52) U.S. Cl. CPC A61F 2/91; A61F 2/915; A61F 2/966; A61F 2250/0003; A61F 2250/0098; A61F 2250/0071; A61F 2210/0014; A61F 2210/0076; A61F 2220/0025

(57) ABSTRACT

An illustrative stent may comprise an elongated tubular body having a scaffolding forming a plurality of cells. The elongated tubular body may extend between a first end and a second end. An inflatable jacket defining an enclosed inflation chamber may surround at least a portion of the scaffolding. The stent may further include an inflation valve.

### CROSS REFERENCE TO RELATED APPLICATIONS

The present application claims the benefit of and priority to U.S. Provisional Patent Application Ser. No. 62/490,538, filed Apr. 26, 2017, the disclosure of which is incorporated herein by reference.

### TECHNICAL FIELD

The present disclosure pertains to medical devices, and methods for manufacturing medical devices. More particularly, the present disclosure pertains to an inflatable stent.

### BACKGROUND

Implantable stents are devices that are placed in a body structure, such as a blood vessel or body cavity, to provide support and to maintain the structure open. These devices are manufactured by any one of a variety of different manufacturing methods and may be used according to any one of a variety of methods. Of the known medical devices, delivery system, and methods, each has certain advantages and disadvantages. There is an ongoing need to provide alternative medical devices and delivery devices as well as alternative methods for manufacturing and using medical devices and delivery devices.

### BRIEF SUMMARY

This disclosure provides design, material, manufacturing method, and use alternatives for medical devices.

In a first example, a stent may comprise an elongated tubular body having a scaffolding forming a plurality of cells, the scaffolding of the elongated tubular body extending continuously between a first end and a second end of the stent, an inflatable jacket defining an enclosed inflation chamber surrounding at least a portion of the scaffolding, and an inflation valve.

Alternatively or additionally to any of the examples above, in another example, the inflatable jacket may include a first inflatable region positioned adjacent to the first end of the stent and a second inflatable region positioned adjacent to the second end of the stent.

Alternatively or additionally to any of the examples above, in another example, the first inflatable region and the second inflatable region may be fluidly coupled through an inflation lumen.

Alternatively or additionally to any of the examples above, in another example, the first inflatable region and the second inflatable region each may comprise an annular balloon.

Alternatively or additionally to any of the examples above, in another example, the inflatable jacket may extend from the first end to the second of the stent.

Alternatively or additionally to any of the examples above, in another example, the inflatable jacket may have an undulating outer surface forming a plurality of peaks and valleys.

Alternatively or additionally to any of the examples above, in another example, the inflatable jacket may be secured to the scaffolding adjacent to at least one of the plurality of valleys.

Alternatively or additionally to any of the examples above, in another example, the inflatable jacket may comprise a unitary inflation chamber.

Alternatively or additionally to any of the examples above, in another example, the inflatable jacket may comprise a plurality of inflation chambers.

Alternatively or additionally to any of the examples above, in another example, the inflatable jacket may be coupled to the scaffolding through one or more links.

Alternatively or additionally to any of the examples above, in another example, the inflation valve may be disposed adjacent to one of the first or second end of the stent.

Alternatively or additionally to any of the examples above, in another example, the inflation valve may be a one-way valve.

Alternatively or additionally to any of the examples above, in another example, an inner wall of the inflatable jacket may be embedded in at least a portion of the scaffolding.

Alternatively or additionally to any of the examples above, in another example, a first end region of the elongated tubular body adjacent to the first end and/or a second end region of the elongated tubular body adjacent to the second end may have an outer diameter larger than an outer diameter of an intermediate region disposed between the first end region and the second end region.

Alternatively or additionally to any of the examples above, in another example, the elongated tubular body may have a uniform outer diameter from the first end to the second end.

In another example, a stent may comprise an elongated tubular body having a scaffolding forming a plurality of cells, the scaffolding of the elongated tubular body extending continuously between a first end and a second end of the stent, an inflatable jacket defining an enclosed inflation chamber surrounding at least a portion of the scaffolding, and an inflation valve.

Alternatively or additionally to any of the examples above, in another example, the to inflatable jacket may include a first inflatable region positioned adjacent to the first end of the stent and a second inflatable region positioned adjacent to the second end of the stent.

Alternatively or additionally to any of the examples above, in another example, the first inflatable region and the second inflatable region may be fluidly coupled through an inflation lumen.

Alternatively or additionally to any of the examples above, in another example, the first inflatable region and the second inflatable region each may comprise an annular balloon.

Alternatively or additionally to any of the examples above, in another example, the inflatable jacket may extend from the first end to the second of the stent.

Alternatively or additionally to any of the examples above, in another example, the inflatable jacket may have an undulating outer surface forming a plurality of peaks and valleys.

Alternatively or additionally to any of the examples above, in another example, the inflatable jacket may be secured to the scaffolding adjacent to at least one of the plurality of valleys.

Alternatively or additionally to any of the examples above, in another example, the inflation valve may be disposed adjacent to one of the first or second end of the stent.

Alternatively or additionally to any of the examples above, in another example, the inflatable jacket may be coupled to the scaffolding through one or more links.

Alternatively or additionally to any of the examples above, in another example, an inner wall of the inflatable jacket may be embedded in at least a portion of the scaffolding.

In another example, a stent may comprise an elongated tubular body having a scaffolding forming a plurality of cells, the scaffolding of the elongated tubular body extending between a first end and a second end of the stent, an inflatable jacket surrounding at least a portion of the scaffolding, the inflatable jacket including a first inflation chamber positioned adjacent to the first end and a second inflation chamber positioned adjacent to the second end, and a first inflation valve.

Alternatively or additionally to any of the examples above, in another example, the first inflation chamber and the second inflation chamber each may comprise an inflatable annular balloon.

Alternatively or additionally to any of the examples above, in another example, the first inflation chamber and the second inflation chamber may be fluidly coupled.

Alternatively or additionally to any of the examples above, in another example, a first end region of the elongated tubular body adjacent to the first end and/or a second end region of the elongated tubular body adjacent to the second end may have an outer diameter larger than an outer diameter of an intermediate region disposed between the first end region and the second end region.

Alternatively or additionally to any of the examples above, in another example, the stent may further comprise a second inflation valve wherein the first inflation valve is in fluid communication with the first inflation chamber and the second inflation valve is in fluid communication with the second inflation valve.

In another example, a stent may comprise an elongated tubular body having a scaffolding forming a plurality of cells, the scaffolding of the elongated tubular body extending continuously between a first end and a second end of the stent, an inflatable jacket surrounding the scaffolding and extending from the first end to the second end of the stent, the inflatable jacket including an inflation chamber having an undulating outer surface, and an inflation valve.

Alternatively or additionally to any of the examples above, in another example, the undulating outer surface of the inflatable jacket may comprise a plurality of peaks and valleys.

Alternatively or additionally to any of the examples above, in another example, the inflatable jacket may be coupled to the scaffolding adjacent to at least one of the plurality of valleys.

Alternatively or additionally to any of the examples above, in another example, the elongated tubular body may have a uniform outer diameter from the first end to the second end.

Alternatively or additionally to any of the examples above, in another example, the inflation valve may be a one-way valve or a break away valve.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments in connection with the accompanying drawings, in which:

FIG. 1 is a side view of an illustrative inflatable stent;

FIG. 2 is a cross-sectional view of the illustrative stent of FIG. 1 taken at line 2-2 of FIG. 1;

FIG. 3 is a side view of an illustrative stent delivery system;

FIG. 4A is a partial cross-sectional view of the illustrative stent of FIG. 1 deployed in a lumen;

FIG. 4B is another partial cross-sectional view of the illustrative stent of FIG. 1 deployed in a lumen;

FIG. 5 is a partial cross-sectional view of another illustrative inflatable stent in a first configuration, deployed in a lumen;

FIG. 6 is a partial cross-sectional view of the illustrative stent of FIG. 5 in a second configuration, deployed in a lumen

FIG. 7 is a side view of another illustrative inflatable stent;

FIG. 8 is a cross-sectional view of the illustrative stent of FIG. 7 taken at line 8-8 of FIG. 7; and

FIG. 9 is a cross-sectional view of another illustrative stent.

While the disclosure is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

In some instances, it may be desirable to provide an endoluminal implant, or stent, that can deliver luminal patency in patients with esophageal strictures. Such stents may be used in patients experiencing dysphagia, sometime due to esophageal cancer. An esophageal stent may allow a patient to maintain nutrition via oral intake during cancer treatment or palliation periods. Covered woven, braided, or knitted stents may prevent tissue ingrowth through the stent cells and may facilitate removal of the stent, if needed. However, covered stents may be more prone to migration than uncovered or partially covered stents. It may be desirable to provide a stent that prevents tissue ingrowth, facilitates removal of the stent, and reduces migration of the stent. While the embodiments disclosed herein are discussed with reference to esophageal stents, it is contemplated that the stents described herein may be used and sized for use in other locations such as, but not limited to: bodily tissue, bodily organs, vascular lumens, non-vascular lumens and combinations thereof, such as, but not limited to, in the coronary or peripheral vasculature, trachea, bronchi, colon, small intestine, biliary tract, urinary tract, prostate, brain, stomach and the like.

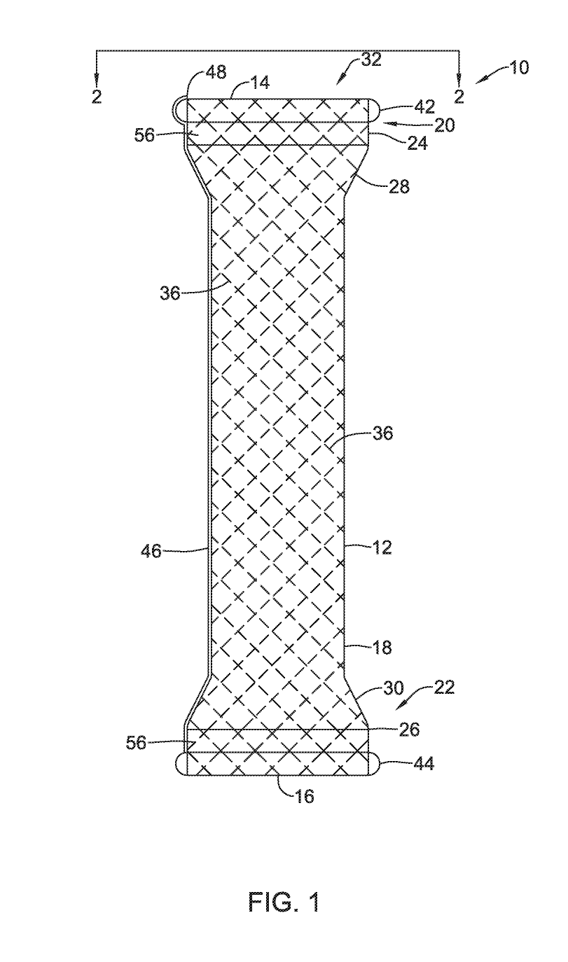

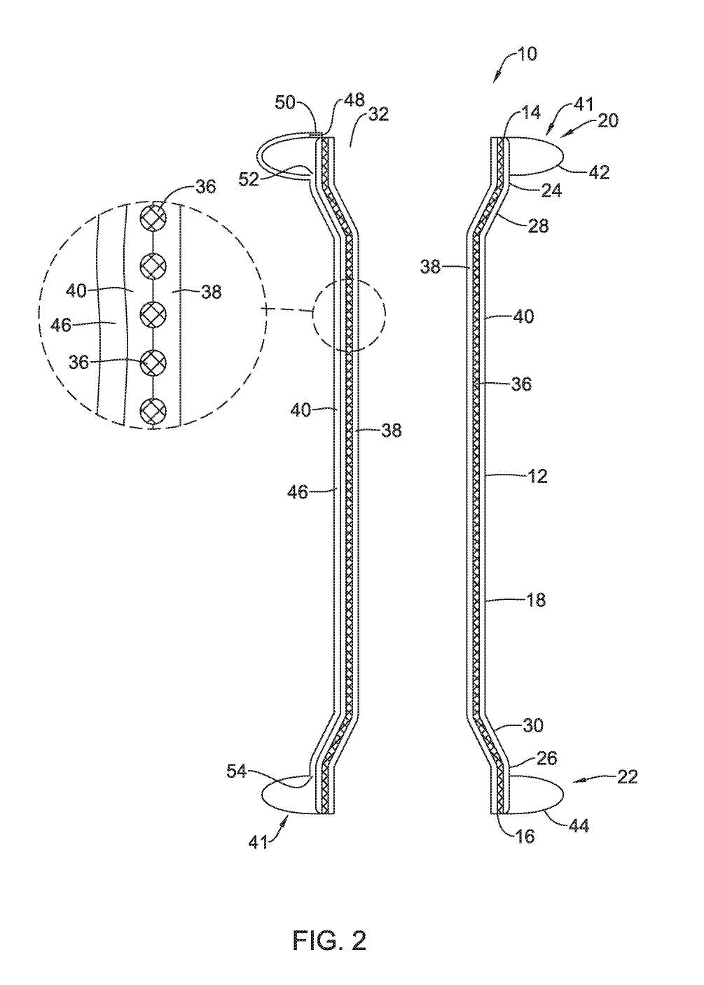

FIG. 1 illustrates a side view of an illustrative endoluminal implant 10, such as, but not limited to, a stent. FIG. 2 illustrates a cross-sectional view of the illustrative stent 10 of FIG. 1, taken at line 2-2. In some instances, the stent 10 may be formed from an elongated tubular member 12. While the stent 10 is described as generally tubular, it is contemplated that the stent 10 may take any cross-sectional shape desired. The stent 10 may have a first, or proximal, end 14, a second, or distal, end 16, and an intermediate region 18 disposed between the first end 14 and the second end 16. The stent 10 may include a lumen 32 extending from a first opening adjacent the first end 14 to a second opening adjacent to the second end 16 to allow for the passage of food, fluids, etc.

The stent 10 may be expandable from a first collapsed configuration (not explicitly shown) to a second expanded configuration, as shown in FIGS. 1 and 2. The stent 10 may be structured to extend across a stricture and to apply a radially outward pressure to the stricture in a lumen to open the lumen and allow for the passage of foods, fluids, air, etc.

The stent 10 may have a scaffold structure, fabricated from a number of filaments or struts 36. The scaffold structure may extend from the first end 14 to the second end 16 of the stent 10. For example, the scaffold structure may extend continuously from the first end 14 to the second end 16 of the stent 10. In some embodiments, the stent 10 may be braided with one filament to form the scaffold structure. In other embodiments, the stent 10 may be braided with several filaments to form the scaffold structure, as is found, for example, in the WallFlex®, WALLSTENT®, and Polyflex® stents, made and distributed by Boston Scientific, Corporation. In another embodiment, the stent 10 may be knitted to form the scaffold structure, such as the Ultraflex™ stents made by Boston Scientific, Corporation. In yet another embodiment, the stent 10 may be of a knotted type, such the Precision Colonic™ stents made by Boston Scientific, Corporation. Thus, in such instances one or more of the filament(s) forming the scaffold structure may extend continuously from the first end 14 to the second end 16 of the stent 10. In still another embodiment, the stent 10 may include a laser cut tubular member to form the scaffold structure, such as the EPIC™ stents made by Boston Scientific, Corporation. A laser cut tubular member may have an open and/or closed cell geometry including one or more interconnected struts. In such instances, the laser cut tubular member forming the scaffold structure may extend continuously from the first end 14 to the second end 16 of the stent 10.

In some instances, an inner and/or outer surface of the scaffold structure of the stent 10 may be entirely, substantially or partially, covered with a polymeric covering or layer 38, 40 (see, for example, FIG. 2). For example, a covering or coating which may help reduce food impaction and/or reduce tumor or tissue ingrowth. In some instances, the inner layer 38 and the outer layer 40 may be formed as a unitary structure. In other embodiments, the inner layer 38 and the outer layer 40 may be formed as separate layers. The inner and outer layers 38, 40 may be formed from the same material or different materials, as desired. The inner layer 38 and/or outer layer 40 may span or be disposed within openings or interstices defined between adjacent stent filaments or struts 36 of the scaffold structure, as more clearly shown in FIG. 2. It can be appreciated that as inner layer 38 and outer layer 40 extend outwardly and inwardly, respectively, they may touch and/or form an interface region within the spaces (e.g., openings, cells, interstices) in the wall of the scaffold structure of the stent 10. For example, the detailed view of FIG. 2 shows that both the inner and outer layers 38, 40 may extend into the openings defined between adjacent stent struts 36 and form an interface region. Further, the inner and outer layers 38, 40 may additionally extend between adjacent filaments or struts 36, thereby filling any space between adjacent filament or strut members 36.

It is contemplated that the stent 10 can be made from a number of different materials such as, but not limited to, metals, metal alloys, shape memory alloys and/or polymers, as desired, enabling the stent 10 to be expanded into shape when accurately positioned within the body. In some instances, the material may be selected to enable the stent 10 to be removed with relative ease as well. For example, the stent 10 can be formed from alloys such as, but not limited to, nitinol and Elgiloy®. Depending on the material selected for construction, the stent 10 may be self-expanding or require an external force to expand the stent 10. In some embodiments, filaments may be used to make the stent 10, which may be composite filaments, for example, having an outer shell made of nitinol having a platinum core. It is further contemplated the stent 10 may be formed from polymers including, but not limited to, polyethylene terephthalate (PET).

In some instances, in the expanded configuration, the stent 10 may include a first end region 20 extending to the first end 14 and a second end region 22 extending to the second end 16. In some embodiments, the first end region 20 and the second end region 22 may include flared regions 24, 26 positioned adjacent to the first end 14 and the second end 16 of the stent 10. The flared regions 24, 26 may be configured to engage an interior portion of the walls of the esophagus, although this is not required. In some embodiments, the flared regions 24, 26 may have a larger diameter than an intermediate region 18 of the stent 10 located between the end regions 20, 22 to prevent or help prevent the stent 10 from migrating once placed in the esophagus or other body lumen. It is contemplated that the transition 28, 30 from the cross-sectional area of the intermediate region 18 to the retention features or flared regions 24, 26 may be gradual, sloped, or occur in an abrupt step-wise manner, as desired.

In some embodiments, the first anti-migration flared region 24 may have a first outer diameter and the second anti-migration flared region 26 may have a second outer diameter. In some instances, the first and second outer diameters may be approximately the same, while in other instances, the first and second outer diameters may be different. In some embodiments, the stent 10 may include only one or none of the flared regions 24, 26. For example, the first end region 20 may include a flare 24 while the second end region 22 may have an outer diameter similar to the intermediate region 18. It is further contemplated that the second end region 22 may include a flare 26 while the first end region 20 may have an outer diameter similar to an outer diameter of the intermediate region 18. In some embodiments, the stent 10 may have a uniform outer diameter from the first end 14 to the second end 16. In some embodiments, the outer diameter of the intermediate region 18 may be in the range of 15 to 25 millimeters. The outer diameter of the flares 24, 26 may be in the range of 20 to 30 millimeters. It is contemplated that the outer diameter of the stent 10 may be varied to suit the desired application.

In some embodiments, the stent 10 may include an inflatable jacket 41 defining an enclosed inflation chamber and surrounding at least a portion of an outer perimeter of the stent 10. In some embodiments, the inflatable jacket 41 may be formed from a high pressure material, such as, but not limited to, polyethylene terephthalate (PET), nylon, polyethylene (PE), polyurethane, or flexible polyvinyl chloride (PVC). In other embodiments, the inflatable jacket 41 may be formed from a compliant, low pressure material, such as, but not limited to, silicone, synthetic polyisoprene, or latex. It is contemplated that the inflatable jacket 41 may be used with a bare stent (e.g., scaffolding only, no liners), a fully covered stent (i.e., a stent having an inner and/or outer covering extending about the entire circumference and along the entire length of the stent scaffolding), or a partially covered stent (i.e., a stent having a covering extending about less than the entire circumference and/or along less than the entire length of the stent scaffolding, leaving a portion of the stent scaffolding exposed for tissue ingrowth through interstices of the scaffolding), as desired. In some embodiments, the inflatable jacket 41 may extend the entire length of the stent 10, from the proximal end 14 to the distal end 16. In other embodiments, the jacket 41 may surround only a portion or a plurality of discrete portions of the length of the stent 10. The inflatable jacket 41 may have a first expandable or inflatable region or chamber 42 positioned adjacent to the proximal end 14 and a second expandable or inflatable region or chamber 44 positioned adjacent to the distal end 16. Each of the first and second inflatable regions or chambers 42, 44 may extend entirely around a circumference of the stent 10, if desired. The tubular scaffolding of the stent 10 may extend radially inward of the first and second inflatable regions or chambers 42, 44 such that the tubular scaffolding extends longitudinally beyond the first and second inflatable regions or chambers 42, 44. For example, the scaffolding may extend the entire length of the stent 10, with the first and second inflatable regions or chambers 42, 44 surrounding and positioned radially outward of the scaffolding.

While inflatable jacket 41 is illustrated as including two inflatable regions 42, 44, it is contemplated that the jacket 41 may include any number of inflatable or expandable regions desired, such as, but not limited to, one, two, three, four, five, or more. It is further contemplated that the inflatable or expandable regions may be positioned anywhere along the length of the stent 10 and spaced evenly or eccentrically, as desired. In some cases, the inflatable jacket 41 may be formed as a unitary structure. In other embodiments, the inflatable jacket 41 may be formed as a plurality of individual structures which may be inflated through a single inflation port or through a plurality of inflation ports, as desired.

The first and second inflatable regions 42, 44 may be configured to be moved between a first collapsed (e.g., deflated) configuration (not explicitly shown) and a second expanded (e.g., inflated) configuration, as shown in FIGS. 1 and 2. An inflation fluid may be provided to the first and/or second inflatable regions 42, 44 through an inflation lumen 46. The inflation fluid may be saline, a biocompatible liquid polymer, such as ENTERYX®, air, gel, or other suitable inflation fluid. The inflation lumen 46 may fluidly couple the first inflatable region 42 and the second inflatable region 44, although this is not required. The inflation lumen 46 may extend along an exterior of the tubular scaffolding of the stent 10 between the first and second inflatable regions 42, 44, if desired. In other instances, the inflation lumen 46 may extend interior of the tubular scaffolding of the stent 10. In some embodiments, the stent 10 may include two or more separate inflation lumens to individually inflate the two or more inflatable regions 42, 44.

In some cases, the first inflatable region 42 and/or the second inflatable region 44 may be a generally rounded annular or ring-like balloon extending about a circumference or an outer perimeter of the stent 10. However, the inflatable regions 42, 44 may take any shape desired. It is further contemplated that the first inflatable region 42 and the second inflatable region 44 need not take the same shape. In some embodiments, the first and/or second inflatable regions 42, 44 may not extend around the entire circumference of the stent 10. In some embodiments, the first and/or second inflatable regions 42, 44 may be formed from two or more circumferentially and/or longitudinally spaced inflatable balloons or other expandable elements.

An inflation port or valve 48 may be positioned adjacent to the first end 14. However, in some instances, the inflation valve 48 may be positioned adjacent to the second end 16 or adjacent to the intermediate region 18, as desired. The inflation valve 48 may be in fluid communication with the inflation lumen 46 to provide a regulated passage for an inflation fluid to travel into the inflatable regions 42, 44 of the stent 10. The inflatable regions 42, 44 may be fluidly coupled to the inflation lumen 46 via openings or valves 52, 54, as desired. In some instances, the valves 52, 54 may be configured to open at different pressure thresholds to permit fluid to enter the first inflatable region 42 and the second inflatable region 44, respectively. For example, the valve 54 of the second inflatable region 44 may open at a lower pressure than the valve 52 of the first inflatable region 42, permitting the second, or distal inflatable region 44 to be at least partially inflated before the valve 52 of the first inflatable region 42 opens to begin inflating the first inflatable region 42. Alternatively, the valve 52 of the first inflatable region 42 may open at a lower pressure than the valve 54 of the second inflatable region 44, permitting the first, or proximal inflatable region 42 to be at least partially inflated before the valve 54 of the second inflatable region 44 opens to begin inflating the second inflatable region 44.

The inflation valve 48 may be any of a number of widely applied valves, applicable in surgeries and medical implants, and may be made from a biocompatible material. In some embodiments, the inflation valve 48 may be a unidirectional, or one-way, valve that provides a regulated passage for an amount of a suitable fluid into the inflation lumen 46 of the stent 10. For example, the inflation valve 48 may provide such a passage upon an application of pressure from a catheter lumen or an inflation device that is introduced into the stent 10 for the stent's inflation. Once the application of pressure is removed, a diaphragm or other sealing mechanism 50 may seal the inflation lumen 46 to maintain the inflatable regions 42, 44 in the inflated state.

The stent 10 may further include one or more radiopaque marker elements 56. The marker elements 56 may be doped with, made of, or otherwise include a radiopaque material. Radiopaque materials are understood to be materials capable of producing a relatively bright image on a fluoroscopy screen or another imaging technique during a medical procedure. The marker elements 56 may be positioned at any location along the length of the stent 10 desired. In some instances, the marker elements 56 may be positioned adjacent to one or both of the first or second end regions 20, 22 to facilitate positioning of the anti-migration inflatable regions 42, 44. This is just an example.

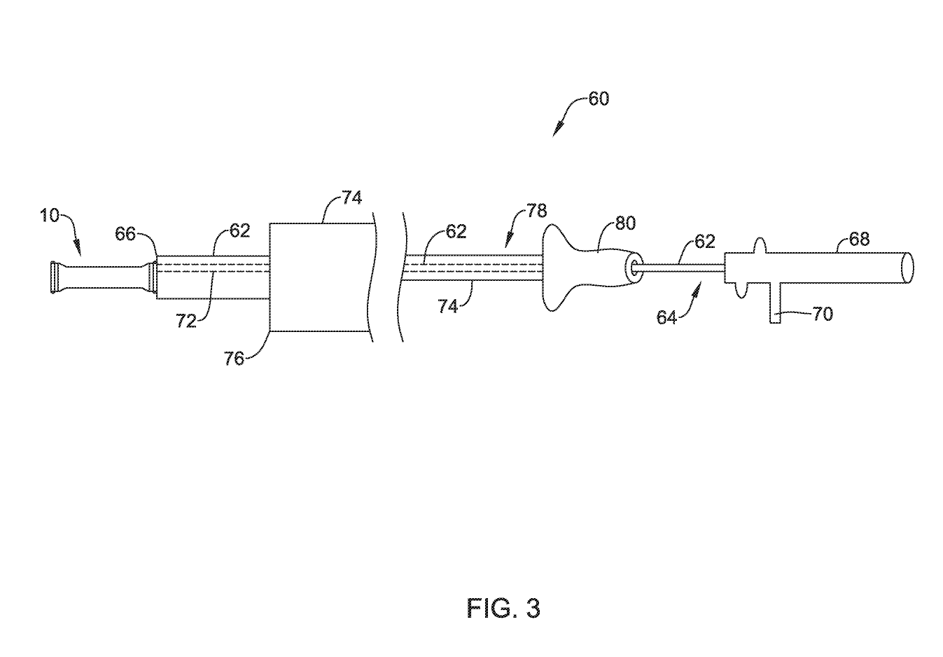

FIG. 3 is a side view of an illustrative stent delivery system 60 for delivering an inflatable stent, such as stent 10. The stent delivery system 60 may include an elongate catheter or pusher shaft 62 having a proximal end region 64 and a distal end 66. The catheter shaft 62 may extend proximally from the distal end 66 to the proximal end region 64 which is configured to remain outside of a patient's body. The proximal end region 64 of the catheter shaft 62 may include a hub 68 attached thereto for connecting other treatment devices or for providing a port for facilitating other treatments. In some embodiments, the hub 68 may include a port 70 or other interface for providing an inflation fluid to the inflatable jacket 41 and/or the one or more of the first or second inflatable regions 42, 44 of the stent 10. It is contemplated that the stiffness and size of the catheter shaft 62 may be modified to form a delivery system 60 for use in various locations within the body. The catheter shaft 62 may further define a lumen 72 through which an inflation fluid may be passed. The lumen 72 may be fluidly coupled at a proximal end thereof to the inflation port 70. A distal portion of the lumen 72 may be configured to releasably couple (fluidly and/or mechanically) to the inflation valve 48 of the stent 10. While not explicitly shown, the catheter shaft 62 may include one or more additional lumens through which a guidewire (not explicitly shown) may be passed in order to advance the catheter to a predetermined position, although this is not required.

The catheter shaft 62 may be configured to be advanced through a working channel of an endoscope, gastroscope, guide sheath, or other guide means 74. The guide sheath 74 may extend proximally from the distal end 76 to the proximal end region 78 which is configured to remain outside of a patient's body. The proximal end region 78 of the guide sheath 74 may include a hub 80 attached thereto for connecting other treatment devices.

The stent 10 may be releasably coupled or secured to the catheter shaft 62 at the inflation valve 48. For example, the inflation valve 48 may be disposed within and/or secured to the inflation lumen 72. As discussed above, the inflation valve 48 may comprise a break away port. The valve 48 may be affixed to the catheter shaft 62 during delivery and inflation of the stent 10 and break away from the catheter shaft 62 and/or stent 10 once the stent 10 has been inflated to a desired pressure. In other embodiments, the stent 10, in the collapsed or uninflated state, may be wrapped around a distal portion of the catheter shaft 62. Other mechanisms for releasably securing the stent 10 to the catheter shaft 62 are contemplated.

During delivery of the stent 10, the guide sheath 74 may be positioned across or adjacent to the desired location of the stent 10. The catheter shaft 62, including the stent 10, may be advanced through a lumen of the guide sheath 74 or otherwise deployed from the guide sheath 74. In some instances, the inflatable stent 10 may be wrapped, folded, or otherwise collapsed and/or compressed while the stent 10 is being advanced to the target location within the guide sheath 74. The guide sheath 74 may help maintain the stent 10 in the collapsed position while the stent 10 is advanced to the target location. Once the stent 10 is near the target location, the catheter shaft 62 may be distally advanced from the guide sheath 74, or the guide sheath 74 may be proximally retracted, to advance the stent 10 from within the guide sheath 74. In some instances, the tubular scaffold of the stent 10 may self-expand in the body lumen once unconstrained by the guide sheath 74, or the tubular scaffold of the stent 10 may be partially (or at least partially) expanded in the body lumen such as with a balloon once deployed from the guide sheath 74. In some instances, the tubular scaffold may be sized such that the tubular scaffold does not push the stent 10 against and/or appreciably apply a radially outward force against the luminal wall of the body lumen (e.g., esophagus) when fully radially expanded therein. Once the stent 10 is in the desired position, an inflation fluid may be supplied through the inflation lumen 72 to the inflation valve 48 and into stent inflation lumen 46 to inflate or expand the first and/or second inflatable regions 42, 44 of the stent 10. The flow of inflation fluid may continue until the first and/or second inflatable regions 42, 44 of the stent 10 achieve a desired pressure and/or inflation state. Inflation of the first and/or second inflatable regions 42, 44 of the stent 10 engages the stent 10 against the luminal wall of the body lumen (e.g., esophagus), thereby exerting a radially outward force against the luminal wall of the body lumen sufficient to anchor the stent 10 within the body lumen to prevent migration of the stent 10 within the body lumen. The first and second inflatable regions 42, 44 may be inflated simultaneously, or the first and second inflatable regions 42, 44 may be inflated sequentially, if desired.

It is contemplated that the pressure of the inflation fluid may be sufficient to deform a sealing mechanism, such as sealing mechanism 50, within the valve 48 to allow fluid to flow into the stent inflation lumen 46. Once the flow of fluid is stopped, the sealing mechanism 50 may close, thus maintaining the first and/or second inflatable regions 42, 44 of the stent 10 in the inflated or expanded state. The catheter shaft 62 and the guide sheath 74 may be removed from the body while leaving the stent 10 at the desired location in the body. It is contemplated that the radial force of the stent 10 and/or anti-migration first and/or second inflatable regions 42, 44 may be sufficient to maintain the stent 10 in the desired location. In some embodiments, the stent 10 may be sutured to the esophagus to further secure the stent 10 in the desired location.

It is contemplated that the stent 10 may be removable after a desired time frame, such as, in the range of: one year, less than one year, less than 9 months, less than 6 months, or less than 3 months. In some instances, the application of a mechanical force may allow the sealing mechanism 50 of the valve 48 to deform and release some of the inflation fluid. In some cases, the device used to inflate the first and/or second inflatable regions 42, 44 may also be used to deflate the first and/or second inflatable regions 42, 44. It is contemplated that the inflation fluid may be removed from the body through aspiration or other mechanism, or allowed to be absorbed into or pass through the body. The release of some of the inflation fluid may reduce the pressure of the inflatable stent 10 thus allowing for easier removal from the body lumen. In some instances, the stent 10 may be removed from the body lumen with the inflatable regions 42, 44 in the deflated state while the scaffold remains in a radially expanded state. Removal from the body may also be facilitated by the inner and/or outer layers 38, 40 which may limit or prevent tissue ingrowth into the scaffolding of the stent 10. Additionally, or alternatively, the first and/or second inflatable regions 42, 44 may be deflated or partially deflated to reposition the stent 10. It is contemplated that the stent 10 may be removed from the body or relocated using devices, such as, but not limited to a lasso loop, rat-tooth jaws, forceps, or other grasping or retrieval mechanisms.

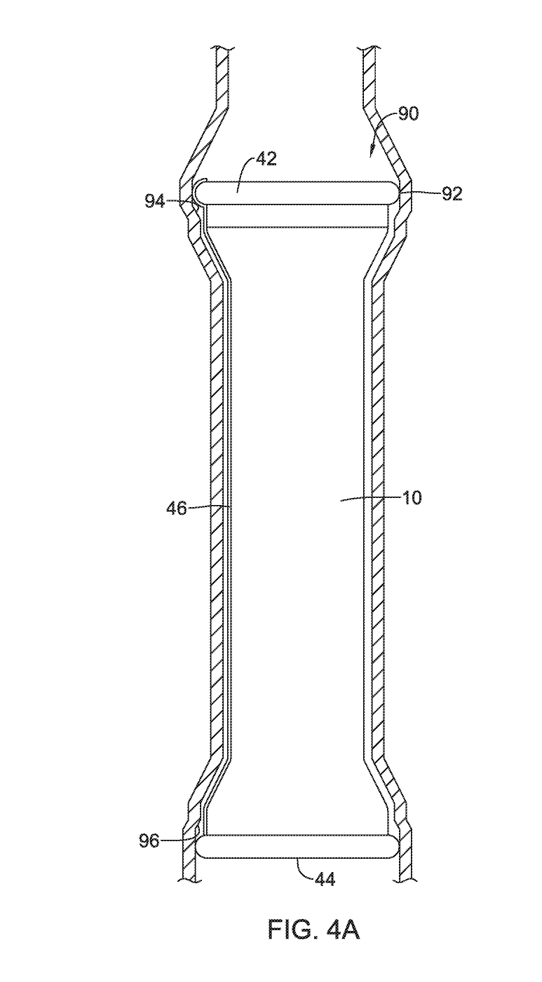

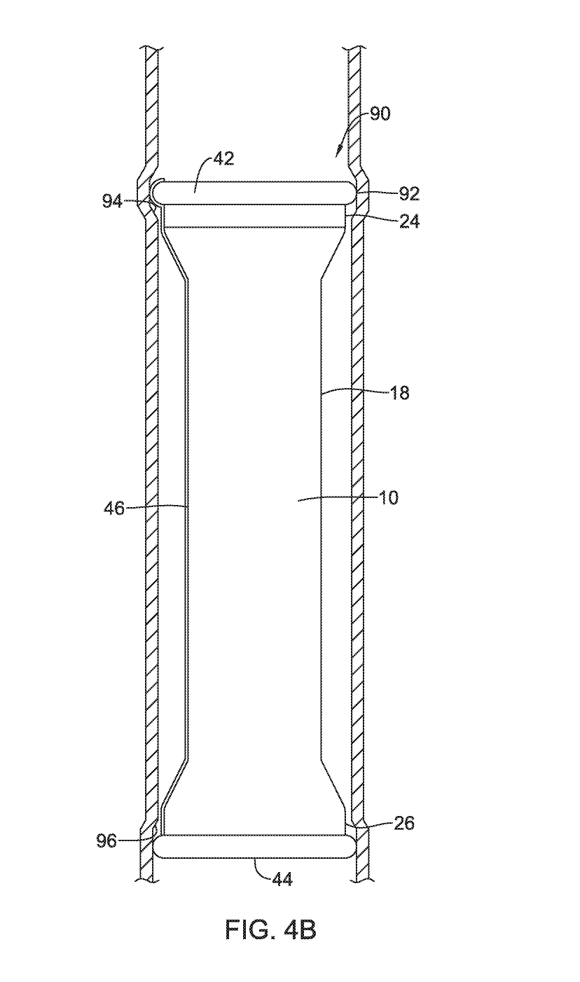

FIG. 4A illustrates a partial cross-sectional view of the illustrative stent 10 deployed in an esophagus 90 in a first exemplary scenario. In the embodiment shown in FIG. 4A, the esophagus 90 has an inner diameter, at the place of implantation, that is smaller than an outer diameter of the illustrative stent 10 along an entire length thereof. As such, the illustrative stent 10 may apply an outward radial force to the esophageal wall 92 along an entire length of the stent 10. As the first and/or second inflatable regions 42, 44 are expanded, they may engage an inner surface of the esophageal wall 92. The first and/or second inflatable regions 42, 44, in the expanded or inflated state, may anchor the stent 10 to the esophageal wall 92 without injuring the wall 92. It is further contemplated that the esophageal wall 92 may conform to the first and/or second inflatable regions 42, 44. This may create a first natural stop 94 distal to the first inflatable region 42 which may limit distal migration of the stent 10 and a second natural stop 96 proximal to the second inflatable region 44 which may limit proximal migration of the stent 10.

FIG. 4B illustrates a partial cross-sectional view of the illustrative stent 10 deployed in an esophagus 90 in a second exemplary scenario. In the embodiment shown in FIG. 4B, the esophagus 90 has an inner diameter, at the place of implantation, that is larger than the intermediate region 18 of the stent 10, but smaller than an outer diameter of the inflatable regions 42, 44 of the illustrative stent 10 in their inflated state. As such, the illustrative stent 10 may apply an outward radial force to the esophageal wall 92 along a region adjacent to the inflatable regions 42, 44 but not contact the esophageal wall 92 along the intermediate region 18 and/or flared regions 24, 26. As the first and/or second inflatable regions 42, 44 are expanded, they may engage an inner surface of the esophageal wall 92. The first and/or second inflatable regions 42, 44, in the expanded or inflated state, may anchor the stent 10 to the esophageal wall 92 without injuring the wall 92. It is further contemplated that the esophageal wall 92 may conform to the first and/or second inflatable regions 42, 44. This may create a first natural stop 94 distal to the first inflatable region 42 which may limit distal migration of the stent 10 and a second natural stop 96 proximal to the second inflatable region 44 which may limit proximal migration of the stent 10.

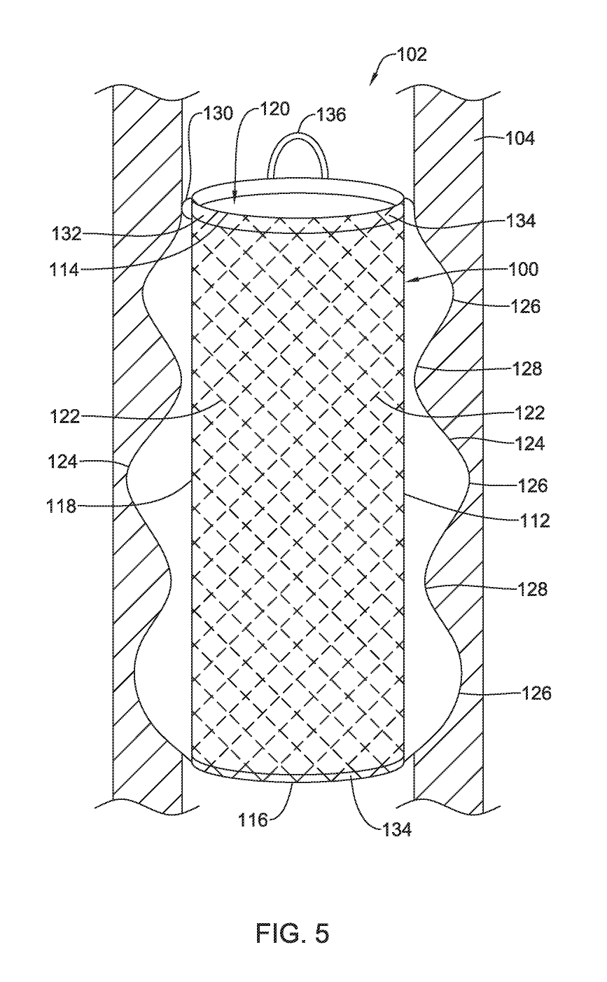

FIG. 5 illustrates a partial cross-sectional view of another endoluminal implant 100, such as, but not limited to, a stent, deployed in an esophagus 102 having an esophageal wall 104. The stent 100 may be similar in form and function to the stent 10 described above. In some instances, the stent 100 may be formed from an elongated tubular member 112. While the stent 100 is described as generally tubular, it is contemplated that the stent 100 may take any cross-sectional shape desired. The stent 100 may have a first, or proximal, end 114, a second, or distal, end 116, and an intermediate region 118 disposed between the first end 114 and the second end 116. The stent 100 may include a lumen 120 extending from a first opening adjacent the first end 114 to a second opening adjacent to the second end 116 to allow for the passage of food, fluids, etc. While the stent body 112 is illustrated as having a generally uniform cross-sectional area (e.g., diameter) from the first end 114 to the second end 116, it is contemplated that the stent 100 may include varying diameter regions. For example, the stent 100 may include flared end regions similar to the flared end regions 24, 26 described above.

The stent 100 may be expandable from a first collapsed configuration (not explicitly shown) to a second expanded configuration, as shown in FIG. 5. The stent 100 may be structured to extend across a stricture and to apply a radially outward pressure to the stricture in a lumen to open the lumen and allow for the passage of foods, fluids, air, etc.

The stent 100 may have a scaffold structure, fabricated from a number of filaments or struts 122. The scaffold structure may extend from the first end 114 to the second end 116 of the stent 100. For example, the scaffold structure may extend continuously from the first end 114 to the second end 116 of the stent 100. In some embodiments, the stent 100 may be braided with one filament to form the scaffold structure. In other embodiments, the stent 100 may be braided with several filaments to form the scaffold structure, as is found, for example, in the WallFlex®, WALLSTENT®, and Polyflex® stents, made and distributed by Boston Scientific, Corporation. In another embodiment, the stent 100 may be knitted to form the scaffold structure, such as the Ultraflex™ stents made by Boston Scientific, Corporation. In yet another embodiment, the stent 100 may be of a knotted type, such the Precision Colonic™ stents made by Boston Scientific, Corporation. Thus, in such instances one or more of the filament(s) forming the scaffold structure may extend continuously from the first end 114 to the second end 116 of the stent 100. In still another embodiment, the stent 100 may include a laser cut tubular member to form the scaffold structure, such as the EPIC™ stents made by Boston Scientific, Corporation. A laser cut tubular member may have an open and/or closed cell geometry including one or more interconnected struts. In such instances, the laser cut tubular member forming the scaffold structure may extend continuously from the first end 114 to the second end 116 of the stent 100.

In some instances, an inner and/or outer surface of the scaffold structure of the stent 100 may be entirely, substantially or partially, covered with a polymeric covering or layer similar in form and function to the inner and/or outer layers 38, 40 described above. For example, a covering or coating which may help reduce food impaction and/or tumor or tissue ingrowth may be reduced. In some instances, the inner layer and the outer layer may be formed as a unitary structure. In other embodiments, the inner layer and the outer layer may be formed as separate layers. The inner and outer layers may be formed from the same material or different materials, as desired. The inner layer and/or outer layer may span or be disposed within openings or interstices defined between adjacent stent filaments or struts 122 of the scaffold structure. In some embodiments, either one of or both of the inner and/or outer layers may not be present.

It is contemplated that the stent 100 can be made from a number of different materials such as, but not limited to, metals, metal alloys, shape memory alloys and/or polymers, as desired, enabling the stent 100 to be expanded into shape when accurately positioned within the body. In some instances, the material may be selected to enable the stent 100 to be removed with relative ease as well. For example, the stent 100 can be formed from alloys such as, but not limited to, nitinol and Elgiloy®. Depending the on material selected for construction, the stent 100 may be self-expanding or require an external force to expand the stent 100. In some embodiments, filaments may be used to make the stent 100, which may be composite filaments, for example, having an outer shell made of nitinol having a platinum core. It is further contemplated the stent 100 may be formed from polymers including, but not limited to, polyethylene terephthalate (PET).

In some embodiments, the stent 100 may include an expandable jacket 124 positioned over and/or surrounding the body 112 of the stent 100 about the circumference thereof. However, in some instances, the jacket 124 may extend around less than the entire circumference of the stent body 112. In some embodiments, the jacket 124 may be formed from a plurality of circumferentially and/or longitudinally spaced inflatable balloons or other expandable elements while in other embodiments, the jacket 124 may be a unitary structure. The jacket 124 may be provided over a bare stent (e.g. no inner and/or outer layers covering the scaffolding or struts 122), a fully covered stent (i.e., a stent having a covering extending about the entire circumference and along the entire length of the stent scaffolding), or a partially covered stent (i.e., a stent having a covering extending about less than the entire circumference and/or along less than the entire length of the stent scaffolding, leaving a portion of the stent scaffolding exposed for tissue ingrowth through interstices of the scaffolding), as desired. In some cases the stent body 112 and the jacket 124 may be formed as a unitary structure.

The jacket 124 may generally take the form of an inflatable or expandable balloon positioned over or surrounding an outer surface of the scaffolding defining the stent body 112. In some embodiments, the jacket 124 may extend over the entire length of the stent 100 while in other embodiments, the jacket 124 may extend over only a portion or a plurality of portions of the length of the stent 100. The jacket 124 may be formed such that an outer surface of the jacket 124 includes one or more peaks 126 and one or valleys 128, relative to a longitudinal axis of the stent 100, along a length of the stent 100 when in the inflated or expanded configuration. In other words, the jacket 124 may have an undulating or wave-like radially outward surface configured to contact a luminal surface of a body lumen when in the expanded configuration. In some cases, the jacket 124 may be secured to the struts 122, or scaffold, adjacent to the valley regions 128 to help form the undulating shape, although this is not required. For example, the jacket 124 may be secured to the scaffolding or a covering overlaying the scaffolding at one or more circumferential attachment regions extending circumferentially around the stent body 112. The undulating shape of the jacket 124 may divide the inflation pressure along a length of the esophageal wall which may reduce localized pressure on the esophageal wall. Further, the undulating shape of the jacket 124 may also provide a plurality of mechanical stops (in combination with the esophageal wall) to reduce stent migration in the proximal and/or distal directions. In some cases, the jacket 124 may also reduce or prevent tissue in-growth.

While the jacket 124 is illustrated as a unitary inflation chamber, it is contemplated that the jacket 124 may be formed from a plurality of individual inflatable chambers. For example, a plurality of ring shaped inflatable chambers may be provided. The plurality of individual chambers may be fluidly coupled such that they may be inflated through a single inflation port. Alternatively or additionally more than one inflation port may be provided such that one or more of the individual inflatable chambers may be inflated separately from one or more of the remaining inflatable chambers.

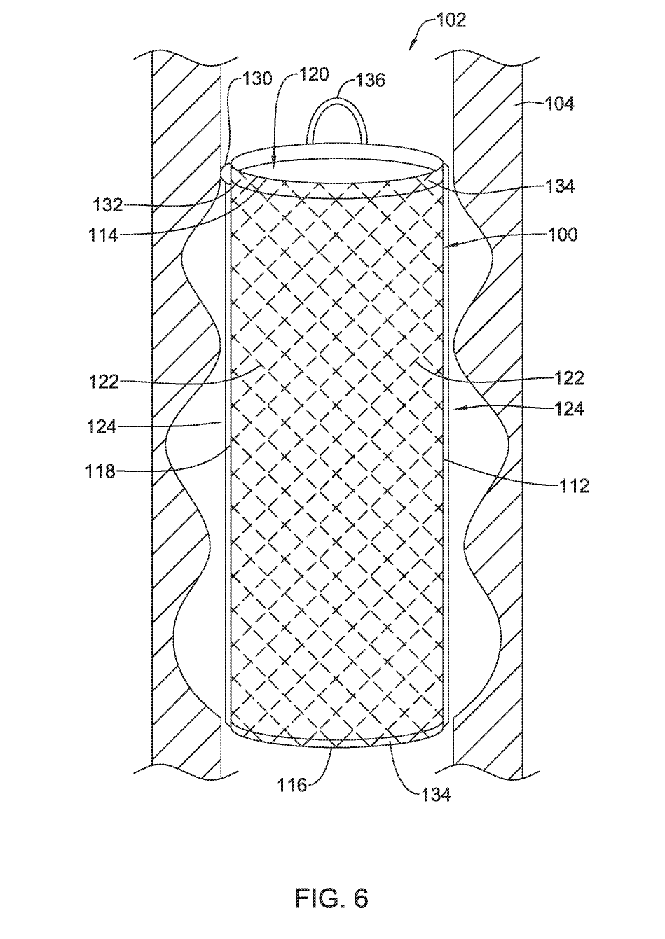

The jacket 124 may be configured to be moved between a first collapsed configuration (see, for example, FIG. 6) and a second expanded configuration, as shown in FIG. 5. An inflation fluid (e.g., air, saline, etc.) may be provided to the jacket 124 through an inflation port 130. The inflation port or valve 130 may be positioned adjacent to the first end 114. However, in some instances, the inflation valve 130 may be positioned adjacent to the second end 116 or adjacent to the intermediate region 118, as desired. The inflation valve 130 may be in fluid communication with the interior of jacket 124 to provide a regulated passage for an inflation fluid to travel into the jacket 124 of the stent 100. The inflation valve 130 may be any of a number of widely applied valves, applicable in surgeries and medical implants, and may be made from a biocompatible material. In some embodiments, the inflation valve 130 may be a unidirectional, or one-way, valve that provides a regulated passage for an amount of a suitable fluid into the jacket 124 of the stent 100. For example, the inflation valve 130 may provide such a passage upon an application of pressure from a catheter lumen or an inflation device that is introduced into the stent 100 for the stent's inflation. Once the application of pressure is removed, a diaphragm or other sealing mechanism 132 may seal the inflation lumen 46 to maintain the jacket 124 in the inflated state.

The stent 100 may further include one or more radiopaque marker elements 134. The marker elements 134 may be doped with, made of, or otherwise include a radiopaque material. Radiopaque materials are understood to be materials capable of producing a relatively bright image on a fluoroscopy screen or another imaging technique during a medical procedure. The marker elements 134 may be positioned at any location along the length of the stent 100 desired. In some instances, the marker elements 134 may be positioned adjacent to one or both of the first or second ends 114, 116 to facilitate positioning of the stent 100. This is just an example.

The stent 100 may be delivered and inflated in a similar manner to the stent 10 as described with respect to FIG. 4. Generally, the stent 100 may be advanced into the esophagus and placed adjacent to the stricture. In some instances, the tubular scaffold of the stent 100 may self-expand in the body lumen once unconstrained by a delivery device. In some instances, the tubular scaffold may be sized such that the tubular scaffold does not push the stent 100 against and/or appreciably apply a radially outward force against the luminal wall of the body lumen (e.g., esophagus) when fully radially expanded therein. Once the stent 100 is in place, the jacket 124 can be inflated and the delivery system removed. Inflation of the jacket 124 of the stent 100 engages the stent 100 against the luminal wall of the body lumen (e.g., esophagus), thereby exerting a radially outward force against the luminal wall of the body lumen sufficient to anchor the stent 100 within the body lumen to prevent migration of the stent 100 within the body lumen. When it is desired to remove the stent 100, the jacket 124 can be deflated, as shown in FIG. 6. With the jacket 124 deflated, the stent 100 may be removed from the body lumen. For instance, the stent 100 may be removed from the body lumen in the deflated state while the scaffold remains in a radially expanded state. In some cases, the stent 100 may be provided with one or more retrieval loops 136 to provide a grasping point for removing the stent 100 and/or at least partially collapsing the scaffold prior to removing the stent 100 from the body lumen.

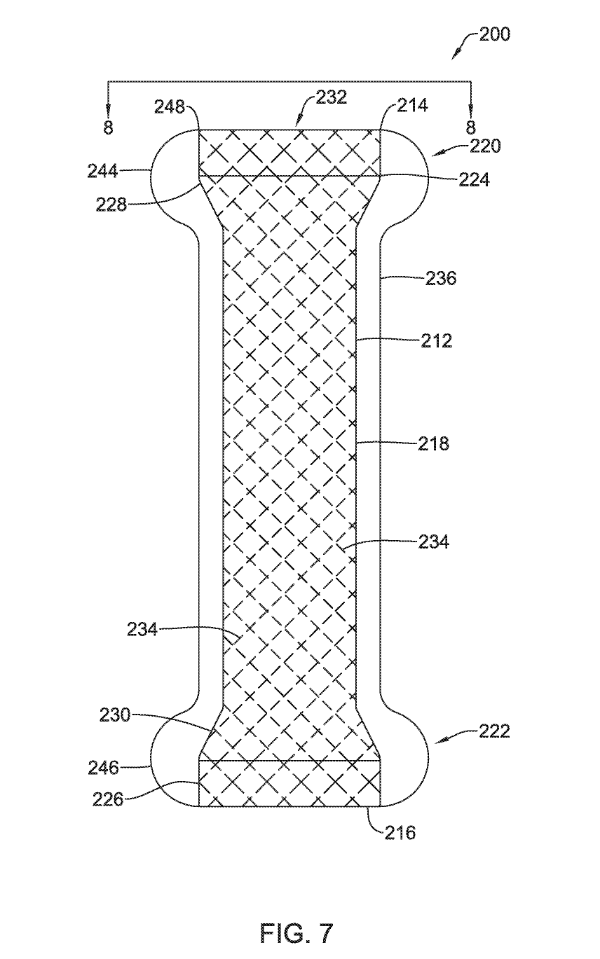

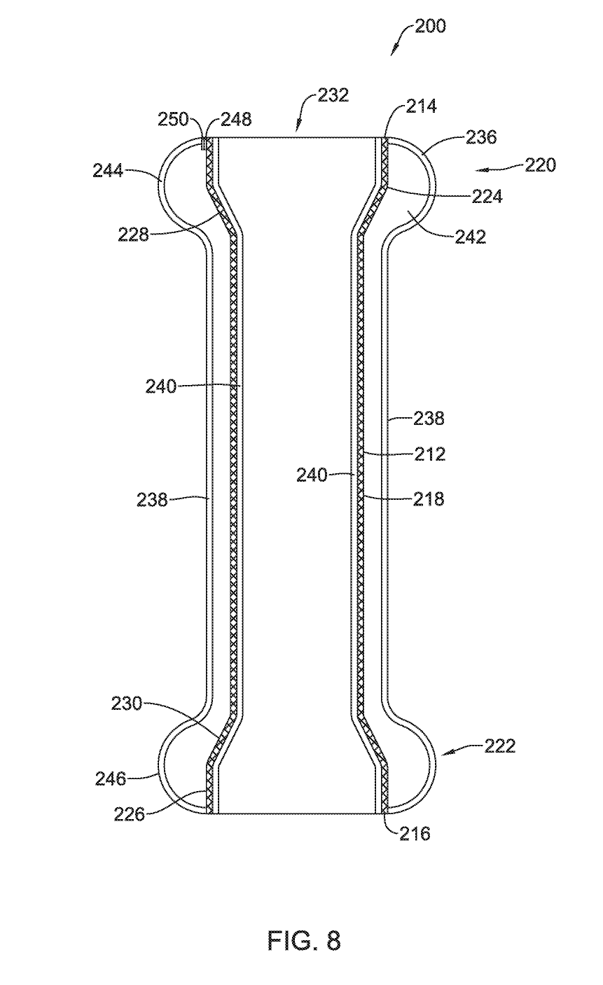

FIG. 7 illustrates a side view of another endoluminal implant 200, such as, but not limited to, a stent. FIG. 8 illustrates a cross-sectional view of the illustrative stent 200 of FIG. 7, taken at line 8-8. The stent 200 may be similar in form and function to the stent 10 described above. In some instances, the stent 200 may be formed from an elongated tubular member 212. While the stent 200 is described as generally tubular, it is contemplated that the stent 200 may take any cross-sectional shape desired. The stent 200 may have a first, or proximal, end 214, a second, or distal, end 216, and an intermediate region 218 disposed between the first end 214 and the second end 216. The stent 200 may include a lumen 232 extending from a first opening adjacent the first end 214 to a second opening adjacent to the second end 216 to allow for the passage of food, fluids, etc.

In some instances, in the expanded configuration, the stent 200 may include a first end region 220 extending to the first end 214 and a second end region 222 extending to the second end 216. In some embodiments, the first end region 220 and the second end region 222 may include flared regions 224, 226 positioned adjacent to the first end 214 and the second end 216 of the stent 200. The flared regions 224, 226 may be configured to engage an interior portion of the walls of the esophagus, although this is not required. In some embodiments, the flared regions 224, 226 may have a larger diameter than an intermediate region 218 of the stent 200 located between the end regions 220, 222 to prevent or help prevent the stent 10 from migrating once placed in the esophagus or other body lumen. It is contemplated that the transition 228, 230 from the cross-sectional area of the intermediate region 218 to the retention features or flared regions 224, 226 may be gradual, sloped, or occur in an abrupt step-wise manner, as desired.

The stent 200 may be expandable from a first collapsed configuration (not explicitly shown) to a second expanded configuration. The stent 200 may be structured to extend across a stricture and to apply a radially outward pressure to the stricture in a lumen to open the lumen and allow for the passage of foods, fluids, air, etc.

The stent 200 may have a scaffold structure, fabricated from a number of filaments or struts 234. The scaffold structure may extend from the first end 214 to the second end 216 of the stent 200. For example, the scaffold structure may extend continuously from the first end 214 to the second end 216 of the stent 200. In some embodiments, the stent 200 may be braided with one filament to form the scaffold structure. In other embodiments, the stent 200 may be braided with several filaments to form the scaffold structure, as is found, for example, in the WallFlex®, WALLSTENT®, and Polyflex® stents, made and distributed by Boston Scientific, Corporation. In another embodiment, the stent 200 may be knitted to form the scaffold structure, such as the Ultraflex™ stents made by Boston Scientific, Corporation. In yet another embodiment, the stent 200 may be of a knotted type, such the Precision Colonic™ stents made by Boston Scientific, Corporation. Thus, in such instances one or more of the filament(s) forming the scaffold structure may extend continuously from the first end 214 to the second end 216 of the stent 200. In still another embodiment, the stent 200 may include a laser cut tubular member to form the scaffold structure, such as the EPIC™ stents made by Boston Scientific, Corporation. A laser cut tubular member may have an open and/or closed cell geometry including one or more interconnected struts. In such instances, the laser cut tubular member forming the scaffold structure may extend continuously from the first end 214 to the second end 216 of the stent 200.

In some instances, an inner and/or outer surface of the scaffold structure of the stent 200 may be entirely, substantially or partially, covered with a polymeric covering or layer similar in form and function to the inner and/or outer layers 38, 40 described above. For example, a covering or coating which may help reduce food impaction and/or tumor or tissue ingrowth may be reduced. In some instances, the inner layer and the outer layer may be formed as a unitary structure. In other embodiments, the inner layer and the outer layer may be formed as separate layers. The inner and outer layers may be formed from the same material or different materials, as desired. The inner layer and/or outer layer may span or be disposed within openings or interstices defined between adjacent stent filaments or struts 234 of the scaffold structure. In some embodiments, either one of or both of the inner and/or outer layers may not be present.

It is contemplated that the stent 200 can be made from a number of different materials such as, but not limited to, metals, metal alloys, shape memory alloys and/or polymers, as desired, enabling the stent 200 to be expanded into shape when accurately positioned within the body. In some instances, the material may be selected to enable the stent 200 to be removed with relative ease as well. For example, the stent 200 can be formed from alloys such as, but not limited to, nitinol and Elgiloy®. Depending the on material selected for construction, the stent 200 may be self-expanding or require an external force to expand the stent 200. In some embodiments, filaments may be used to make the stent 200, which may be composite filaments, for example, having an outer shell made of nitinol having a platinum core. It is further contemplated the stent 200 may be formed from polymers including, but not limited to, polyethylene terephthalate (PET).

In some embodiments, the stent 200 may include an expandable jacket 236 positioned over and/or surrounding the body 212 of the stent 200 about the circumference thereof. However, in some instances, the jacket 236 may extend around less than the entire circumference of the stent body 212. In some embodiments, the jacket 236 may be formed from a plurality of circumferentially and/or longitudinally spaced inflatable balloons or other expandable elements while in other embodiments, the jacket 236 may be a unitary structure. The jacket 236 may be provided over a bare stent (e.g. no inner and/or outer layers covering the scaffolding or struts 234), a fully covered stent (i.e., a stent having a covering extending about the entire circumference and along the entire length of the stent scaffolding), or a partially covered stent (i.e., a stent having a covering extending about less than the entire circumference and/or along less than the entire length of the stent scaffolding, leaving a portion of the stent scaffolding exposed for tissue ingrowth through interstices of the scaffolding), as desired. In some cases the stent body 212 and the jacket 236 may be formed as a unitary structure.

The jacket 236 may generally take the form of an inflatable or expandable balloon positioned over or surrounding an outer surface of the scaffolding defining the stent body 212 and configured to move between an unexpanded and an expanded configuration. The jacket 236 may include an outer wall 238 and an inner wall 240 defining an inflation chamber 242 there between. In some embodiments, the inner wall 240 may be embedded in the scaffolding or struts 234, although this is not required. In some cases, the inner wall 240 may be positioned on an outer surface or an inner surface of the scaffolding, as desired. It is contemplated that if so provided, a stent covering (inner or outer) may form the inner wall 240 of the inflatable jacket 236.

In some embodiments, the jacket 236 may extend over the entire length of the stent 200 while in other embodiments, the jacket 236 may extend over only a portion or a plurality of portions of the length of the stent 200. The jacket 236 may be formed such that an outer surface of the jacket 236 includes a first, or proximal, flared region 244 adjacent to the first end 214 and a second, or distal, flared region 246 adjacent to the second end 216. All, or a portion, of the jacket 236 may be configured to engage an inner surface of a body lumen when the inflatable jacket 236 in the expandable configuration. In some cases, the flared regions 244, 246 in combination with an outward force on the body lumen may provide mechanical stop points for preventing or limiting migration of the stent 200 as described in more detail with respect to FIG. 4 and stent 10.

While the jacket 236 is illustrated as a unitary inflation chamber, it is contemplated that the jacket 236 may be formed from a plurality of individual inflatable chambers. For example, a plurality of ring shaped inflatable chambers may be provided. The plurality of individual chambers may be fluidly coupled such that they may be inflated through a single inflation port. Alternatively or additionally more than one inflation port may be provided such that one or more of the individual inflatable chambers may be inflated separately from one or more of the remaining inflatable chambers.

The jacket 236 may be configured to be moved between a first collapsed configuration (not explicitly shown) and a second expanded configuration, as shown in FIGS. 7 and 8. An inflation fluid (e.g., air, saline, etc.) may be provided to the jacket 236 through an inflation port 248. The inflation port or valve 248 may be positioned adjacent to the first end 214. However, in some instances, the inflation valve 248 may be positioned adjacent to the second end 216 or adjacent to the intermediate region 218, as desired. The inflation valve 248 may be in fluid communication with the interior (e.g., inflation chamber 242) of the jacket 236 to provide a regulated passage for an inflation fluid to travel into the jacket 236 of the stent 200. The inflation valve 248 may be any of a number of widely applied valves, applicable in surgeries and medical implants, and may be made from a biocompatible material. In some embodiments, the inflation valve 248 may be a unidirectional, or one-way, valve that provides a regulated passage for an amount of a suitable fluid into the jacket 236 of the stent 200. For example, the inflation valve 248 may provide such a passage upon an application of pressure from a catheter lumen or an inflation device that is introduced into the stent 200 for the stent's inflation. Once the application of pressure is removed, a diaphragm or other sealing mechanism 250 may seal the inflation valve 248 to maintain the jacket 236 in the inflated state.

The stent 200 may further include one or more radiopaque marker elements (not explicitly shown). The marker elements may be doped with, made of, or otherwise include a radiopaque material. Radiopaque materials are understood to be materials capable of producing a relatively bright image on a fluoroscopy screen or another imaging technique during a medical procedure. The marker elements may be positioned at any location along the length of the stent 200 desired. In some instances, the marker elements may be positioned adjacent to one or both of the first or second ends 214, 216 to facilitate positioning of the stent 200. This is just an example.

The stent 200 may be delivered and inflated in a similar manner to the stent 10 as described with respect to FIG. 4. Generally, the stent 200 may be advanced into the esophagus and placed adjacent to the stricture. In some instances, the tubular scaffold of the stent 200 may self-expand in the body lumen once unconstrained by a delivery device. In some instances, the tubular scaffold may be sized such that the tubular scaffold does not push the stent 200 against and/or appreciably apply a radially outward force against the luminal wall of the body lumen (e.g., esophagus) when fully radially expanded therein. Once the stent 200 is in place, the jacket 236 can be inflated and the delivery system removed. Inflation of the jacket 236 of the stent 200 engages the stent 200 against the luminal wall of the body lumen (e.g., esophagus), thereby exerting a radially outward force against the luminal wall of the body lumen sufficient to anchor the stent 200 within the body lumen to prevent migration of the stent 200 within the body lumen. When it is desired to remove the stent 200, the jacket 236 can be deflated. With the jacket 236 deflated, the stent 200 may be removed from the body lumen. For instance, the stent 200 may be removed from the body lumen in the deflated state while the scaffold remains in a radially expanded state. In some cases, the stent 200 may be provided with one or more retrieval loops to provide a grasping point for removing the stent 200 and/or at least partially collapsing the scaffold prior to removing the stent 200 from the body lumen.

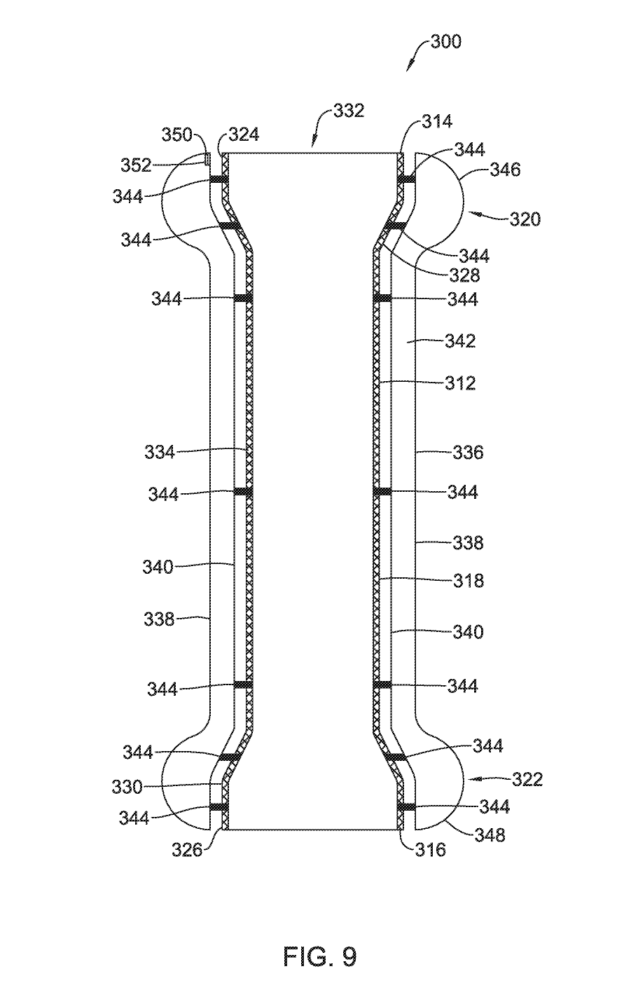

FIG. 9 illustrates a cross-sectional view of another endoluminal implant 300, such as, but not limited to, a stent. The stent 300 may be similar in form and function to the stent 10 described above. In some instances, the stent 300 may be formed from an elongated tubular member 312. While the stent 300 is described as generally tubular, it is contemplated that the stent 300 may take any cross-sectional shape desired. The stent 300 may have a first, or proximal, end 314, a second, or distal, end 316, and an intermediate region 318 disposed between the first end 314 and the second end 316. The stent 300 may include a lumen 332 extending from a first opening adjacent the first end 314 to a second opening adjacent to the second end 316 to allow for the passage of food, fluids, etc.

In some instances, in the expanded configuration, the stent 300 may include a first end region 320 extending to the first end 314 and a second end region 322 extending to the second end 316. In some embodiments, the first end region 320 and the second end region 322 may include flared regions 324, 326 positioned adjacent to the first end 314 and the second end 316 of the stent 300. The flared regions 324, 326 may be configured to engage an interior portion of the walls of the esophagus, although this is not required. In some embodiments, the flared regions 324, 326 may have a larger diameter than an intermediate region 318 of the stent 300 located between the end regions 320, 322 to prevent or help prevent the stent 10 from migrating once placed in the esophagus or other body lumen. It is contemplated that the transition 328, 330 from the cross-sectional area of the intermediate region 318 to the retention features or flared regions 324, 326 may be gradual, sloped, or occur in an abrupt step-wise manner, as desired.

The stent 300 may be expandable from a first collapsed configuration (not explicitly shown) to a second expanded configuration. The stent 300 may be structured to extend across a stricture and to apply a radially outward pressure to the stricture in a lumen to open the lumen and allow for the passage of foods, fluids, air, etc.

The stent 300 may have a scaffold structure, fabricated from a number of filaments or struts 334. The scaffold structure may extend from the first end 314 to the second end 316 of the stent 300. For example, the scaffold structure may extend continuously from the first end 314 to the second end 316 of the stent 300. In some embodiments, the stent 300 may be braided with one filament to form the scaffold structure. In other embodiments, the stent 300 may be braided with several filaments to form the scaffold structure, as is found, for example, in the WallFlex®, WALLSTENT®, and Polyflex® stents, made and distributed by Boston Scientific, Corporation. In another embodiment, the stent 300 may be knitted to form the scaffold structure, such as the Ultraflex™ stents made by Boston Scientific, Corporation. In yet another embodiment, the stent 300 may be of a knotted type, such the Precision Colonic™ stents made by Boston Scientific, Corporation. Thus, in such instances one or more of the filament(s) forming the scaffold structure may extend continuously from the first end 314 to the second end 316 of the stent 300. In still another embodiment, the stent 300 may include a laser cut tubular member to form the scaffold structure, such as the EPIC™ stents made by Boston Scientific, Corporation. A laser cut tubular member may have an open and/or closed cell geometry including one or more interconnected struts. In such instances, the laser cut tubular member forming the scaffold structure may extend continuously from the first end 314 to the second end 316 of the stent 300.

In some instances, an inner and/or outer surface of the scaffold structure of the stent 300 may be entirely, substantially or partially, covered with a polymeric covering or layer similar in form and function to the inner and/or outer layers 38, 40 described above. For example, a covering or coating which may help reduce food impaction and/or tumor or tissue ingrowth may be reduced. In some instances, the inner layer and the outer layer may be formed as a unitary structure. In other embodiments, the inner layer and the outer layer may be formed as separate layers. The inner and outer layers may be formed from the same material or different materials, as desired. The inner layer and/or outer layer may span or be disposed within openings or interstices defined between adjacent stent filaments or struts 334 of the scaffold structure. In some embodiments, either one of or both of the inner and/or outer layers may not be present.

It is contemplated that the stent 300 can be made from a number of different materials such as, but not limited to, metals, metal alloys, shape memory alloys and/or polymers, as desired, enabling the stent 300 to be expanded into shape when accurately positioned within the body. In some instances, the material may be selected to enable the stent 300 to be removed with relative ease as well. For example, the stent 300 can be formed from alloys such as, but not limited to, nitinol and Elgiloy®. Depending the on material selected for construction, the stent 300 may be self-expanding or require an external force to expand the stent 300. In some embodiments, filaments may be used to make the stent 300, which may be composite filaments, for example, having an outer shell made of nitinol having a platinum core. It is further contemplated the stent 300 may be formed from polymers including, but not limited to, polyethylene terephthalate (PET).

In some embodiments, the stent 300 may include an expandable jacket 336 positioned over and/or surrounding the body 312 of the stent 300 about the circumference thereof. However, in some instances, the jacket 336 may extend around less than the entire circumference of the stent body 312. In some embodiments, the jacket 336 may be formed from a plurality of circumferentially and/or longitudinally spaced inflatable balloons or other expandable elements while in other embodiments, the jacket 336 may be a unitary structure. The jacket 336 may be provided over a bare stent (e.g. no inner and/or outer layers covering the scaffolding or struts 334), a fully covered stent (i.e., a stent having a covering extending about the entire circumference and along the entire length of the stent scaffolding), or a partially covered stent (i.e., a stent having a covering extending about less than the entire circumference and/or along less than the entire length of the stent scaffolding, leaving a portion of the stent scaffolding exposed for tissue ingrowth through interstices of the scaffolding), as desired. In some cases the stent body 312 and the jacket 336 may be formed as a unitary structure.

The jacket 336 may generally take the form of an inflatable or expandable balloon positioned over or surrounding an outer surface of the scaffolding defining the stent body 312 and configured to move between an unexpanded and an expanded configuration. The jacket 336 may include an outer wall 338 and an inner wall 340 defining an inflation chamber 342 there between. In some embodiments, the inner wall 340 may be spaced radially outward from the scaffolding or struts 334 (or outer covering of the stent), providing a gap between the inner wall 340 of the jacket 336 and the outer surface of the scaffolding or struts 334 and/or outer covering, although this is not required. The jacket 336 may be coupled to the scaffolding or struts 334 through a plurality of links 344, such as tethers, ties, or lining bands. In some cases, one or more of the links 344 may extend about an entire circumference of the scaffolding 334. In other cases, one or more of the links 344 may be positioned at discrete spaced locations about the circumference and/or length of the stent 300. The links 344 may be formed as a unitary structure with the inflatable jacket 336 and/or the scaffolding 334 (or coverings, if so provided). Alternatively, the links 344 may be coupled (e.g., adhered or otherwise mechanically coupled) to one or both the inflatable jacket 336 and/or the scaffolding 334 (or coverings, if so provided).

In some embodiments, the jacket 336 may extend over the entire length of the stent 300 while in other embodiments, the jacket 336 may extend over only a portion or a plurality of portions of the length of the stent 300. The jacket 336 may be formed such that an outer surface of the jacket 336 includes a first, or proximal, flared region 346 adjacent to the first end 314 and a second, or distal, flared region 348 adjacent to the second end 316. All, or a portion, of the jacket 336 may be configured to engage an inner surface of a body lumen when the inflatable jacket 336 in the expandable configuration. In some cases, the flared regions 346, 348 in combination with an outward force on the body lumen may provide mechanical stop points for preventing or limiting migration of the stent 300 as described in more detail with respect to FIG. 4 and stent 10.

While the jacket 336 is illustrated as a unitary inflation chamber, it is contemplated that the jacket 336 may be formed from a plurality of individual inflatable chambers. For example, a plurality of ring shaped inflatable chambers may be provided. The plurality of individual chambers may be fluidly coupled such that they may be inflated through a single inflation port. Alternatively or additionally more than one inflation port may be provided such that one or more of the individual inflatable chambers may be inflated separately from one or more of the remaining inflatable chambers.

The jacket 336 may be configured to be moved between a first collapsed configuration (not explicitly shown) and a second expanded configuration, as shown in FIG. 9. An inflation fluid (e.g., air, saline, etc.) may be provided to the jacket 336 through an inflation port 350. The inflation port or valve 350 may be positioned adjacent to the first end 314. However, in some instances, the inflation valve 350 may be positioned adjacent to the second end 316 or adjacent to the intermediate region 318, as desired. The inflation valve 350 may be in fluid communication with the interior (e.g., inflation chamber 342) of the jacket 336 to provide a regulated passage for an inflation fluid to travel into the jacket 336 of the stent 300. The inflation valve 350 may be any of a number of widely applied valves, applicable in surgeries and medical implants, and may be made from a biocompatible material. In some embodiments, the inflation valve 350 may be a unidirectional, or one-way, valve that provides a regulated passage for an amount of a suitable fluid into the jacket 336 of the stent 300. For example, the inflation valve 350 may provide such a passage upon an application of pressure from a catheter lumen or an inflation device that is introduced into the stent 300 for the stent's inflation. Once the application of pressure is removed, a diaphragm or other sealing mechanism 352 may seal the inflation valve 350 to maintain the jacket 336 in the inflated state.

The stent 300 may further include one or more radiopaque marker elements (not explicitly shown). The marker elements may be doped with, made of, or otherwise include a radiopaque material. Radiopaque materials are understood to be materials capable of producing a relatively bright image on a fluoroscopy screen or another imaging technique during a medical procedure. The marker elements may be positioned at any location along the length of the stent 300 desired. In some instances, the marker elements may be positioned adjacent to one or both of the first or second ends 314, 316 to facilitate positioning of the stent 300. This is just an example.

The stent 300 may be delivered and inflated in a similar manner to the stent 10 as described with respect to FIG. 4. Generally, the stent 300 may be advanced into the esophagus and placed adjacent to the stricture. In some instances, the tubular scaffold of the stent 100 may self-expand in the body lumen once unconstrained by a delivery device. In some instances, the tubular scaffold may be sized such that the tubular scaffold does not push the stent 300 against and/or appreciably apply a radially outward force against the luminal wall of the body lumen (e.g., esophagus) when fully radially expanded therein. Once the stent 300 is in place, the jacket 336 can be inflated and the delivery system removed. Inflation of the jacket 336 of the stent 300 engages the stent 300 against the luminal wall of the body lumen (e.g., esophagus), thereby exerting a radially outward force against the luminal wall of the body lumen sufficient to anchor the stent 300 within the body lumen to prevent migration of the stent 300 within the body lumen. When it is desired to remove the stent 300, the jacket 336 can be deflated. With the jacket 336 deflated, the stent 300 may be removed from the body lumen. For instance, the stent 300 may be removed from the body lumen in the deflated state while the scaffold remains in a radially expanded state. In some cases, the stent 300 may be provided with one or more retrieval loops to provide a grasping point for removing the stent 300 and/or at least partially collapsing the scaffold prior to removing the stent 300 from the body lumen.

The stents, delivery systems, and the various components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. In some other embodiments, a superelastic alloy, for example a superelastic nitinol can be used to achieve desired properties.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

In some embodiments, a degree of Magnetic Resonance Imaging (MRI) compatibility is imparted into the stents or delivery systems. For example, the stents or delivery systems or portions thereof, may be made of a material that does not substantially distort the image and create substantial artifacts (i.e., gaps in the image). Certain ferromagnetic materials, for example, may not be suitable because they may create artifacts in an Mill image. The stents or delivery systems or portions thereof, may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A stent, the stent comprising:
an elongated tubular body having a scaffolding forming a plurality of cells, the scaffolding of the elongated tubular body extending continuously between a first end and a second end of the stent;
an inflatable jacket defining an enclosed inflation chamber surrounding at least a portion of the scaffolding; and
an inflation valve.

2. The stent of claim 1, wherein the inflatable jacket includes a first inflatable region positioned adjacent to the first end of the stent and a second inflatable region positioned adjacent to the second end of the stent.

3. The stent of claim 2, wherein the first inflatable region and the second inflatable region are fluidly coupled through an inflation lumen.

4. The stent of claim 2, wherein the first inflatable region and the second inflatable region each comprise an annular balloon.

5. The stent of claim 1, wherein the inflatable jacket extends from the first end to the second of the stent.

6. The stent of claim 5, wherein the inflatable jacket has an undulating outer surface forming a plurality of peaks and valleys.

7. The stent of claim 6, wherein the inflatable jacket is secured to the scaffolding adjacent to at least one of the plurality of valleys.

8. The stent of claim 1, wherein the inflation valve is disposed adjacent to one of the first or second end of the stent.

9. The stent of claim 1, wherein the inflatable jacket is coupled to the scaffolding through one or more links.

10. The stent of claim 1, wherein an inner wall of the inflatable jacket is embedded in at least a portion of the scaffolding.

11. A stent, the stent comprising:
an elongated tubular body having a scaffolding forming a plurality of cells, the scaffolding of the elongated tubular body extending continuously between a first end and a second end of the stent;
an inflatable jacket surrounding at least a portion of the scaffolding, the inflatable jacket including a first inflation chamber positioned adjacent to the first end and a second inflation chamber positioned adjacent to the second end; and
a first inflation valve.

12. The stent of claim 11, wherein the first inflation chamber and the second inflation chamber each comprise an inflatable annular balloon.

13. The stent of claim 11, wherein the first inflation chamber and the second inflation chamber are fluidly coupled.

14. The stent of claim 11, wherein a first end region of the elongated tubular body adjacent to the first end and/or a second end region of the elongated tubular body adjacent to the second end has an outer diameter larger than an outer diameter of an intermediate region disposed between the first end region and the second end region.

15. The stent of claim 11, further comprising a second inflation valve wherein the first inflation valve is in fluid communication with the first inflation chamber and the second inflation valve is in fluid communication with the second inflation valve.

16. A stent, the stent comprising:
an elongated tubular body having a scaffolding forming a plurality of cells, the scaffolding of the elongated tubular body extending between a first end and a second end of the stent;
an inflatable jacket surrounding the scaffolding and extending from the first end to the second end of the stent, the inflatable jacket including an inflation chamber having an undulating outer surface; and
an inflation valve.

17. The stent of claim 16, wherein the undulating outer surface of the inflatable jacket comprises a plurality of peaks and valleys.

18. The stent of claim 17, wherein the inflatable jacket is coupled to the scaffolding adjacent to at least one of the plurality of valleys.

19. The stent of claim 16, wherein the elongated tubular body has a uniform outer diameter from the first end to the second end.

20. The stent of claim 16, wherein the inflation valve is a one-way valve or a break away valve.
